(19) United States
(12) Patent Application Publication
(10) Pub. No.: US 2022/0061849 A1
(43) Pub. Date: Mar. 3, 2022

(54) ANVIL ASSEMBLY WITH IMPROVED CUT RING ASSEMBLY

(71) Applicant: Covidien LP, Mansfield, MA (US)

(72) Inventors: Syed Sarfraz Ahamed, Shanghai (CN); Huabiao Yang, Shanghai (CN)

(21) Appl. No.: 17/298,976

(22) PCT Filed: Dec. 6, 2018
(86) PCT No.: PCT/CN2018/119480

§ 371 Date: Jun. 2, 2021

(51) Int. Cl. A61B 17/115

(52) U.S. Cl. CPC A61B 17/1155; A61B 2017/00955

(57) ABSTRACT

An anvil assembly includes a center rod and a head assembly. The center rod defines a longitudinal axis and has a proximal portion and a distal portion. The head assembly includes a housing, a central post supported within the housing, and a cut ring assembly. The housing supports an anvil that defines a plurality of staple deforming pockets. The central post and the housing define an annular cavity. The cut ring assembly is positioned within the annular cavity and includes a backup plate and a cut ring, wherein the backup plate is formed of a thermoplastic polymer and the cut ring is formed of polyethylene and is over molded onto the backup plate.

### BACKGROUND

### 1. Technical Description

The present disclosure is directed to circular stapling devices and, more particularly, to anvil assemblies for circular stapling devices with improved cut ring assemblies.

### 2. Background of Related Art

Circular stapling devices include a staple cartridge that supports annular rows of staples and an anvil assembly that includes an annular anvil surface that is positioned to receive the annular rows of staples to form the staples in tissue. The anvil assembly is movable in relation to the staple cartridge between spaced and clamped positions. The anvil assembly includes an anvil center rod and an anvil head that is supported on a distal portion of the anvil center rod. In some embodiments, the anvil head is pivotable in relation to the anvil center rod from a tilted reduced profile position to an operative position in juxtaposed alignment with the staple cartridge.

In certain circular stapling devices, the anvil head includes a cut ring assembly that is positioned to provide a surface within the anvil head to facilitate cutting of tissue. More specifically, the cut ring assembly is positioned to provide a backup surface for an annular knife to assist the knife in cutting through tissue clamped between the staple cartridge and the anvil assembly. In some anvil assemblies that have a tiltable head, the cut ring assembly is also positioned to retain the anvil head in the operative position until the stapling device is fired.

A continuing need exists in the art for an improved cut ring assembly for an anvil assembly that is low cost and provides a surface within the anvil assembly for effective cutting of tissue.

### SUMMARY

One aspect of the present disclosure is directed to an anvil assembly including a center rod and a head assembly. The center rod defines a longitudinal axis and has a proximal portion and a distal portion. The head assembly includes a housing, a central post supported within the housing, and a cut ring assembly. The housing supports an anvil that defines a plurality of staple deforming pockets. The central post and an outer wall of the housing define an annular cavity. The cut ring assembly is positioned within the annular cavity and includes a backup plate and a cut ring, wherein the backup plate is formed of a thermoplastic polymer and the cut ring is formed of polyethylene and is over molded onto the backup plate.

In some embodiments, the thermoplastic polymer is a polyether ether ketone.

In certain embodiments, the center rod is pivotably coupled to the central post of the head assembly by a pivot member such that the head assembly is movable in relation to the center rod from an operative position to a tilted position.

In embodiments, the cut ring assembly is slidably positioned about the post within the annular cavity and is moveable within the annular cavity from a proximal position to a distal position.

In some embodiments, the cut ring assembly is positioned to engage the center rod when the cut ring assembly is in the proximal position to retain the head assembly in the operative position.

In certain embodiments, the backup plate of the cut ring assembly includes a plurality of legs that extend distally from the backup plate and is positioned to engage the housing to retain the cut ring assembly in the proximal position.

In embodiments, the plurality of legs is configured to fracture when the cut ring assembly moves towards the distal position.

In some embodiments, the backup plate includes at least one radial protrusion and the distal portion of the center rod includes at least one stop surface, wherein the at least one radial protrusion is supported on the at least one stop surface when the cut ring assembly is in the proximal position to retain the head assembly in the operative position.

In certain embodiments, the at least one radial protrusion is spaced from the at least one stop surface when the cut ring assembly is in the distal position.

In embodiments, the at least one radial protrusion includes two radial protrusions and the at least one stop surface includes two stop surfaces.

Another aspect of the present disclosure is directed to a surgical stapling device including an elongate body, a shell assembly, and an anvil assembly. The elongate body has a proximal portion and a distal portion. The shell assembly is supported on the distal portion of the elongate body and includes a staple cartridge that supports an annular array of staples. The anvil assembly includes a center rod and a head assembly. The center rod defines a longitudinal axis and has a proximal portion and a distal portion. The head assembly includes a housing, a central post supported within the housing, and a cut ring assembly. The housing supports an anvil that defines a plurality of staple deforming pockets. The central post and the housing define an annular cavity. The cut ring assembly is positioned within the annular cavity and includes a backup plate and a cut ring, wherein the backup plate is formed of a thermoplastic polymer and the cut ring is formed of polyethylene and is over molded onto the backup plate.

In some embodiments, the stapling device includes a handle assembly that supports the proximal portion of the elongate body.

### BRIEF DESCRIPTION OF THE DRAWINGS

Various embodiments of the presently disclosed anvil assembly including an improved cut ring assembly are described herein below with reference to the drawings, wherein:

FIG. 1 is a side perspective view of a circular stapling device including an exemplary embodiment of the presently disclosed anvil assembly with the anvil assembly in a spaced, operative position in relation to a staple cartridge of the stapling device;

FIG. 2 is a side perspective view of the anvil assembly shown in FIG. 1 with an anvil head of the anvil assembly in an operative position;

FIG. 3 is an exploded view of the anvil assembly shown in FIG. 1;

FIG. 4 is an enlarged view of the indicated area of detail shown in FIG. 3;

FIG. 5 is a perspective view of a cut ring assembly of the anvil assembly shown in FIG. 2;

FIG. 6 is a perspective view from the proximal end of a backup ring of the cut ring assembly shown in FIG. 5;

FIG. 7 is a perspective view from the distal end of the backup ring of the cut ring assembly shown in FIG. 5;

FIG. 8 is a perspective view from the proximal end of the cut ring of the cut ring assembly shown in FIG. 5;

FIG. 9 is an enlarged view of the indicated area of detail shown in FIG. 1;

FIG. 10 is a cross-sectional view taken through a distal portion of the circular stapling device shown in FIG. 1 with the anvil assembly in a clamped, pre-fired, operative position and the distal portion of the stapling device shown in phantom;

FIG. 11 is a cross-sectional view taken through the distal portion of the anvil assembly shown in FIG. 2 with the anvil assembly in a post-fired, tilted position; and

FIG. 12 is a perspective view of the anvil assembly shown in FIG. 11 in the post-fired, tilted position.

### DETAILED DESCRIPTION OF EMBODIMENTS

The presently disclosed anvil assembly will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel.

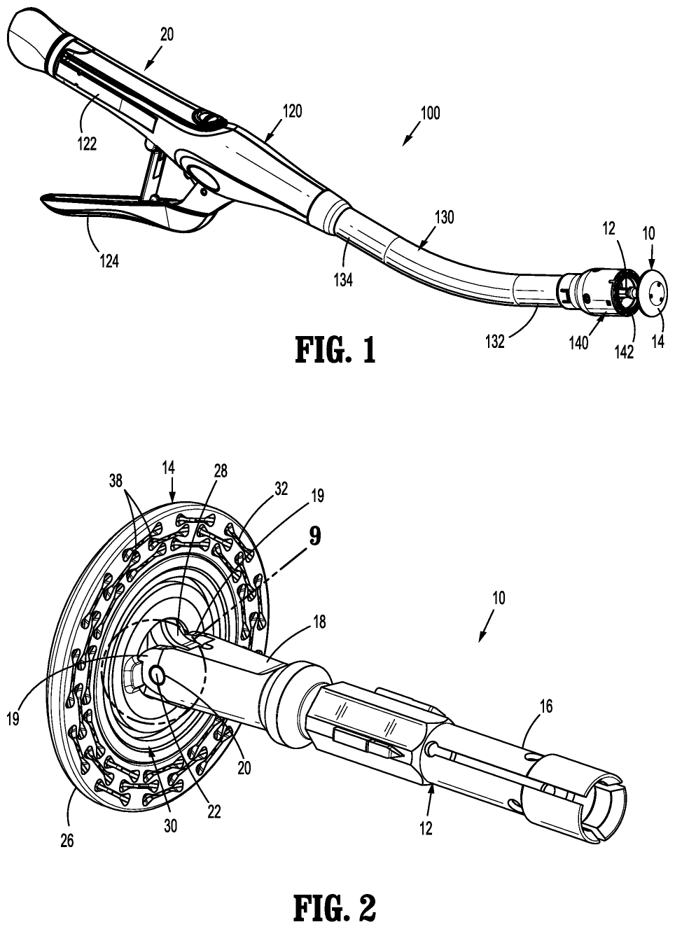

Referring to FIG. 1, the presently disclosed anvil assembly shown generally as anvil assembly 10 is illustrated in association with a circular stapling device shown generally as stapling device 100. The stapling device 100 includes a handle or actuator assembly 120, an elongate body 130, a shell assembly 140, and the anvil assembly 10. The handle assembly 120 as illustrated includes a grip 122 and an actuation trigger or lever 124 that can be depressed to actuate various functions of the stapling device 100 including approximation of the anvil assembly 10 with the shell assembly 140 and firing of staples (not shown). Although the stapling device 100 is illustrated as a manually actuated stapling device, it is envisioned that the presently disclosed anvil assembly 10 can be used with a variety of different types of stapling devices including electrically powered stapling devices and robotic devices.

The elongate body 130 includes a distal portion 132 and a proximal portion 134. The distal portion 132 of the elongate body 130 supports the shell assembly 140. The shell assembly 140 includes a staple cartridge 142 that supports an array of staples (not shown). In some embodiments, the array of staples has a circular configuration and may include two or more circular rows of staples.

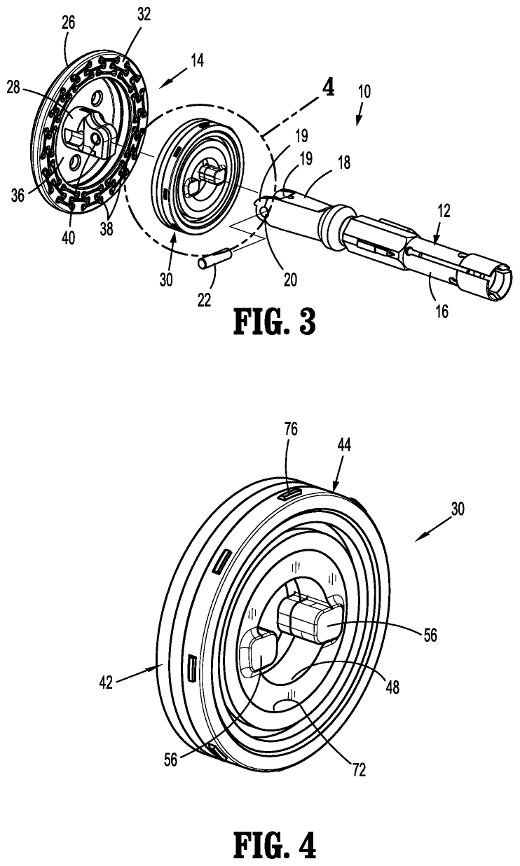

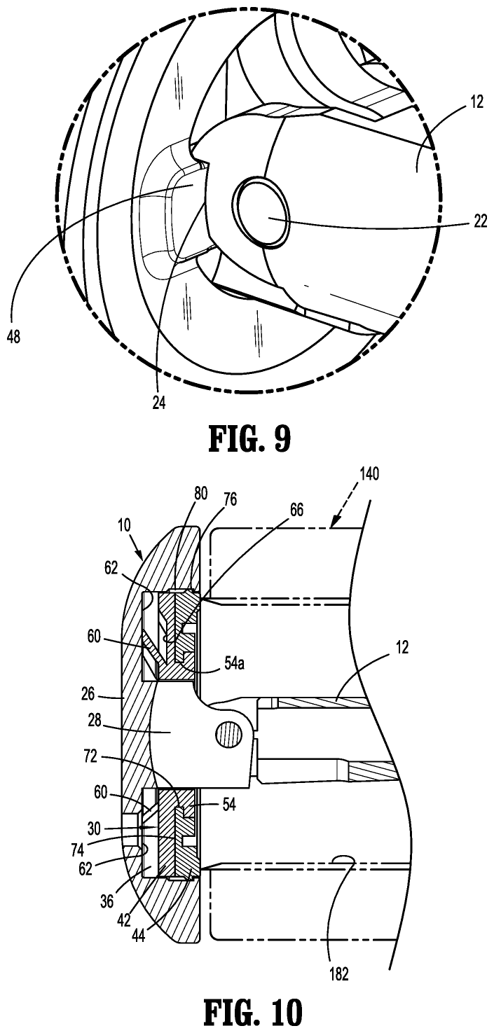

Referring to FIGS. 2 and 3, the presently disclosed anvil assembly 10 includes an anvil center rod 12, and an anvil head assembly 14. The anvil center rod 14 includes a proximal portion 16 and a distal portion 18. The proximal portion 16 of the anvil center rod 12 is adapted to couple the anvil assembly 10 to an anvil retainer (not shown) of the surgical stapling device 100 (FIG. 1) in a releasable fashion. The distal portion 18 of the anvil center rod 12 includes a pair of spaced legs 19 which include a transverse through bore 20 that receives a pivot member 22 to pivotably connect the anvil center rod 12 to the anvil head assembly 14. The distal portion of the anvil center rod 12 also defines stop surfaces 24 (FIG. 9) that are provided to retain the anvil head assembly 14 in an operative position (FIG. 1) until the stapling device 100 is fired as described in further detail below. The configuration of the anvil center rod 12 is similar to that known in the prior art and will not be discussed in further detail herein. For a more detailed description of the anvil center rod 12, see, e.g., U.S. Pat. No. 7,303,106 (“the '106 Patent”) and U.S. Pat. No. 8,348,122 (“the '122 Patent”), which are incorporated herein by reference in their entirety.

The anvil head assembly 14 is pivotably supported on the distal portion 18 of the anvil center rod 12 by the pivot member 22 and includes a housing 26, a post 28, a cut ring assembly 30, and an anvil 32. The housing that receives the post 28 is to define an annular cavity 36 within the recess of the housing 26. The anvil 32 is supported on the housing 26 and is positioned about the annular cavity 36. The anvil 32 defines a plurality of staple deforming pockets 38, each of which is aligned with a respective staple in the staple cartridge 142 (FIG. 1) such that when the staples are fired from the staple cartridge 142, the staples are received within the pockets 38 and deformed. For a more detailed description of the how the stapling device 100 is fired, see the '106 and '122 Patents.

As discussed above, the head assembly 14 is pivotably supported on the distal portion 18 of the center rod 12 of the anvil assembly 10 by the pivot member 22. More specifically, the post 28 of the anvil head assembly 14 is positioned between the spaced legs 19 of the center rod 12 and defines a transverse bore 40 that receives the pivot member 22. The pivot member 22 also extends through the bores 20 in the spaced legs 19 in the distal portion 18 of the center rod 12 such that the head assembly 14 is pivotable on the center rod 12 between an operative position (FIG. 2) and a tilted position (FIG. 12). Although not shown, the anvil head assembly 14 is urged towards the tilted position by a biasing member.

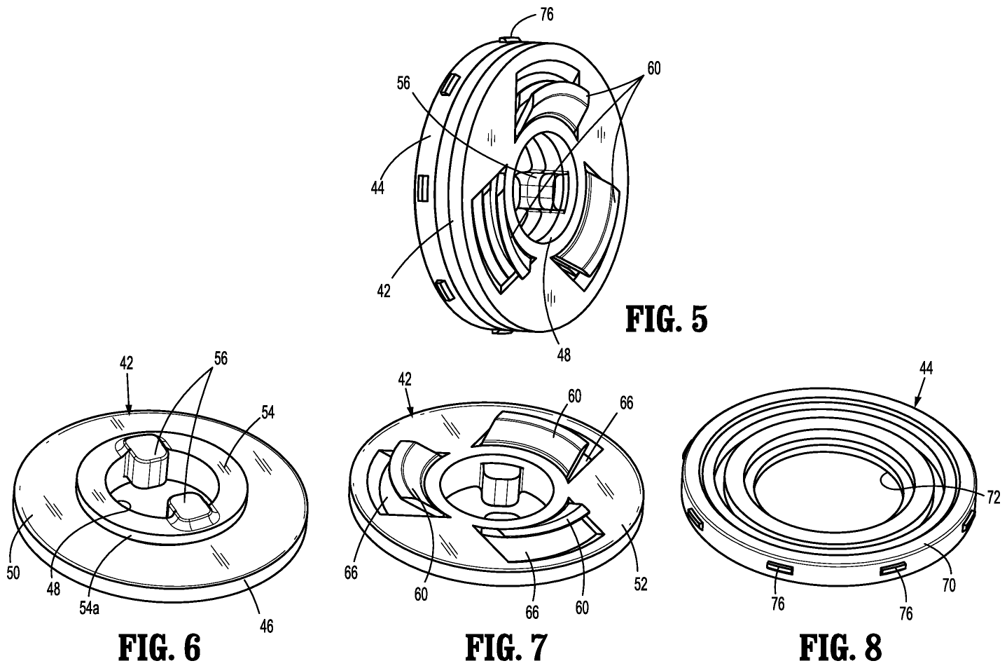

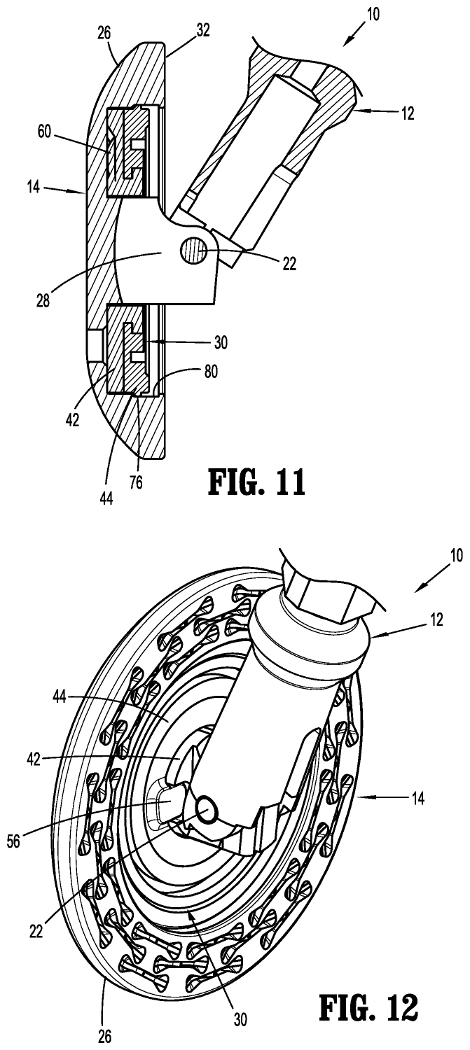

Referring to FIGS. 4-9, the cut ring assembly 30 includes a backup plate 42 and a cut ring 44. The backup plate 42 includes an annular body 46 that defines a central opening 48 that receives the post 28 of the head assembly 14 such that the cut ring assembly 30 is moveable about the post 28 within the annular cavity 36 of the housing 26 from a proximal position (FIG. FIG. 10) to a distal position (FIG. 11). The annular body 46 includes a proximal face 50 (FIG. 6), a distal face 52 (FIG. 7), a raised flange 54 positioned about the central opening 48 on the proximal face 50, and radial protrusions 56 that extend from the raised flange 54 into the central opening 48 of the annular body 46. The raised flange 54 defines an undercut 54a (FIG. 10). The radial protrusions 48 are positioned on the stop surfaces 24 (FIG. 9) of the anvil center rod 12 when the cut ring assembly 30 is in the proximal position to prevent movement of the head assembly 14 from the operative position to the tilted position. The radial protrusions 48 are spaced from the stop surfaces 24 (FIG. 11) of the anvil center rod 12 when the cut ring assembly 30 is in the distal position to allow pivotal movement of the head assembly 14 from the operative to the tilted position.

In embodiments, the distal face 52 (FIG. 7) of the backup plate 42 includes a plurality of legs 60 that extend from the distal face 52 and engage an inner wall 62 (FIG. 10) of the housing 26. The legs 52 are deformable and/or frangible to retain the cut ring assembly 30 in the proximal position (FIG. 10) until a predetermined force in the distal direction is applied to the cut ring assembly 30. When the predetermined force is applied to the cut ring assembly 30, the legs 60 of the backup plate will deform or fracture to allow the cut ring assembly 30 to move to the distal position. The distal face 52 of the backup plate 42 defines pockets 66 (FIG. 7) that receive the legs 60 when the legs 60 are deformed or fracture.

Referring to FIGS. 8-10, the cut ring 44 includes an annular body 70 that defines a central bore 72 that receives the raised flange 54 of the anvil assembly 14 to secure the cut ring 44 onto the backup plate 42. The annular body 70 includes a distal face 74 (FIG. 10) that is received in abutting relation to the proximal face 50 of the backup plate 42. A sidewall of the cut ring 44 includes protrusions 76 (FIG. 8) that are received in a groove 80 formed in the housing 26 to guide movement of the cut ring assembly 30 between the proximal position and the distal position.

In embodiments, the backup plate 42 is formed from a thermoplastic polymer such as polyether ether ketone (PEEK) and the cut ring 44 is formed from a polyethylene material that is over molded onto the thermoplastic polymer. The cost of the anvil assembly 10 can be substantially reduced by forming the backup plate 42 from a thermoplastic polymer such as PEEK rather than a metal such as stainless steel.

Referring to FIGS. 9 and 10, when the cut ring assembly 30 is in the proximal position, the radial protrusions 48 of the backup plate 42 are engaged with the stop surfaces 24 on the distal portion 18 of the anvil center rod 12 to retain the head assembly 14 in the operative position. In this position, the cut ring 44 is aligned with an annular knife 182 (FIG. 10) of the stapling device 100 (FIG. 1).

Referring also to FIGS. 11 and 12, when the stapling device 100 is fired to eject staples from the staple cartridge 142 (FIG. 1), the annular knife 182 (FIG. 10) is advanced into the cut ring assembly 30. When the knife 182 is driven into the cut ring assembly 30, the predetermined force on the cut ring assembly 30 to deform or fracture the legs 60 of the backup plate 42 is exceeded and the cut ring assembly 30 moves within the annular cavity 36 from the proximal position (FIG. 10) to the distal position (FIG. 11). As the cut ring assembly 30 moves from the proximal position (FIG. 10) to the distal position (FIG. 11), the radial protrusions 48 of the backup plate 42 move away from the stop surfaces 24 on the distal portion 18 of the center rod 12 to allow the head assembly 14 of the anvil assembly 10 to pivot from the operative position to the tilted position. As is known in the art, pivoting of the anvil assembly 10 from the operative position to the tilted position will only occur after the anvil assembly 10 is moved in relation to the shell assembly 140 to a spaced or unapproximated position.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. An anvil assembly comprising:
a center rod having a proximal portion and a distal portion and defining a longitudinal axis;
a head assembly including a housing, a central post supported within the housing, and a cut ring assembly, the housing supporting an anvil defining a plurality of staple deforming pockets, the central post and the housing defining an annular cavity, the cut ring assembly being positioned within the annular cavity and including a backup plate and a cut ring, wherein the backup plate is formed of a thermoplastic polymer and the cut ring is formed of polyethylene and is over molded onto the backup plate.

2. The anvil assembly of claim 1, wherein the thermoplastic polymer is a polyether ether ketone.

3. The anvil assembly of claim 1, wherein the center rod is pivotably coupled to the central post of the head assembly by a pivot member, the head assembly being movable in relation to the center rod from an operative position to a tilted position.

4. The anvil assembly of claim 3, wherein the cut ring assembly is slidably positioned about the post within the annular cavity, the cut ring assembly being moveable within the annular cavity from a proximal position to a distal position.

5. The anvil assembly of claim 4, wherein the cut ring assembly is positioned to engage the center rod when the cut ring assembly is in the proximal position to retain the head assembly in the operative position.

6. The anvil assembly of claim 5, wherein the backup plate includes a plurality of legs, the plurality of legs extending distally from the backup plate and being positioned to engage the housing to retain the cut ring assembly in the proximal position.

7. The anvil assembly of claim 6, wherein the plurality of legs are configured to fracture when the cut ring assembly moves towards the distal position.

8. The anvil assembly of claim 5, wherein the backup plate includes at least one radial protrusion and the distal portion of the center rod includes at least one stop surface, the at least one radial protrusion being supported on the at least one stop surface when the cut ring assembly is in the proximal position to retain the head assembly in the operative position.

9. The anvil assembly of claim 8, wherein the at least one radial protrusion is spaced from the at least one stop surface when the cut ring assembly is in the distal position.

10. The anvil assembly of claim 9, wherein the at least one radial protrusion includes two radial protrusions and the at least one stop surface includes two stop surfaces.

11. A surgical stapling device comprising:
an elongate body having a proximal portion and a distal portion;
a shell assembly supported on the distal portion of the elongate body, the shell assembly including a staple cartridge that supports an annular array of staples; and
an anvil assembly including a center rod and a head assembly, the center rod having a proximal portion and a distal portion and defining a longitudinal axis, the head assembly including a housing, a central post supported within the housing, and a cut ring assembly, the housing supporting an anvil that defines a plurality of staple deforming pockets, the central post and the housing defining an annular cavity, the cut ring assembly being positioned within the annular cavity and including a backup plate and a cut ring, wherein the backup plate is formed of a thermoplastic polymer and the cut ring is formed of polyethylene, the cut ring being over molded onto the backup plate.

12. The surgical stapling device of claim 11, wherein the thermoplastic polymer is a polyether ether ketone.

13. The surgical stapling device of claim 11, wherein the center rod is pivotably coupled to the central post of the head assembly by a pivot member, the head assembly being movable from an operative position to a tilted position.

14. The surgical stapling device of claim 13, wherein the cut ring assembly is slidably positioned about the post within the annular cavity, the cut ring assembly being moveable within the annular cavity from a proximal position to a distal position.

15. The surgical stapling device of claim 14, wherein the cut ring is positioned to engage the center rod when the cut ring assembly is in the proximal position to retain the head assembly in the operative position.

16. The surgical stapling device of claim 15, wherein the backup plate includes a plurality of legs, the plurality of legs extending distally from the backup plate and being positioned to engage the housing to retain the cut ring assembly in the proximal position.

17. The surgical stapling device of claim 16, wherein the plurality of legs are configured to fracture when the cut ring assembly moves towards the distal position.

18. The surgical stapling device of claim 15, wherein the backup plate includes at least one radial protrusion and the distal portion of the center rod includes at least one stop surface, the at least one radial protrusion being supported on the at least one stop surface when the cut ring assembly is in the proximal position to retain the head assembly in the operative position.

19. The surgical stapling device of claim 18, wherein the at least one radial protrusion is spaced from the at least one stop surface when the cut ring assembly is in the distal position.

20. The surgical stapling device of claim 19, wherein the at least one radial protrusion includes two radial protrusions and the at least one stop surface includes two stop surfaces.
